(19) United States
(12) Patent Application Publication
(10) Pub. No.: US 2020/0384209 A1
(43) Pub. Date: Dec. 10, 2020

(54) Drive mechanism

(71) Applicant: SHL MEDICAL AG, ZUG (CH)

(72) Inventor: Brian Maxfield, Boca Raton, FL (US)

(21) Appl. No.: 16/992,243

(22) Filed: Aug. 13, 2020

Related U.S. Application Data
(63) Continuation-in-part of application No. 15/564,123, filed on Oct. 3, 2017, now Pat. No. 10,758,677.

(30) Foreign Application Priority Data
Apr. 24, 2015 (SE) 1550497-0

(51) Int. Cl. A61M 5/32; A61M 5/315

(52) U.S. Cl. CPC A61M 5/3204; A61M 2005/3151; A61M 5/31563; A61M 5/31511

(57) ABSTRACT

The present invention relates to a drive mechanism for a medicament delivery device, comprising a plunger rod (54) arranged to act on a stopper (28) arranged in a medicament container (16); a compression drive spring (58) operably connected to said plunger rod (54) and capable of applying a force on said plunger rod (54); a spring guide (78) arranged coaxial with, and radially outside, said compression drive spring (58), capable of preventing buckling of said drive spring, wherein said spring guide (78) comprises a resilient force element (86) capable of applying a force on said medicament container (16).

### CROSS REFERENCE TO RELATED APPLICATIONS

The present application is a continuation-in-part of U.S. application Ser. No. 15/564,123 filed on Oct. 3, 2017, which is a national stage application of international application no. PCT/EP2016/056384 filed on Mar. 23, 2016, which claims priority to Swedish application no. 1550497-0 filed Apr. 24, 2015, the contents of all of which are hereby incorporated by reference.

### TECHNICAL AREA

The present invention relates to a drive mechanism to be used with a medicament delivery device, and in particular a drive mechanism arranged with a compression spring.

### BACKGROUND OF INVENTION

A number of medicament delivery devices have been developed wherein a medicament container is placed inside the housing of the devices. The medicament container has to be supported in an appropriate manner in order to minimize the risk of breakage, which is a pronounced risk when the containers often are made of glass and wherein the force of a power pack, comprising a compression spring, often provide strong forces.

On the other hand, due to manufacturing aspects, as few components as possible are favoured, especially for disposable medicament delivery devices in order to keep the costs as low as possible.

With these aspects in mind some developments have been made. For instance the document US 2007/0021718 discloses the use of a compression spring for elastically supporting a medicament container in place in a housing of a medicament delivery device. The spring may be positioned between a distal end surface of the medicament container and an insert which is secured axially in the housing. The spring is preferably made of a plastic material.

Further, often when compression springs are used, some sort of support is required for preventing buckling of the spring. One common solution is then to have a guide rod extending inside the compression spring, such as is disclosed for example in WO 2013/032389. Another solution is to have a hollow plunger rod that is extra long and to place the compression spring inside the plunger rod.

A drawback with the use of a guide rod is that an extra component is needed, which component is not optimal from an assembly point of view of a power unit, because it has to be attached to a distal end wall of the medicament delivery device. On the other hand, the use of very long plunger rods is not optimal from a design point of view because the medicament delivery devices become long and not very attractive for an end user.

### BRIEF DESCRIPTION OF INVENTION

In the present application, when the term “distal part/end” is used, this refers to the part/end of the device, or the parts/ends of the members thereof, which during use of the device is located the furthest away from the medicament delivery site of the patient. Correspondingly, when the term “proximal part/end” is used, this refers to the part/end of the device, or the parts/ends of the members thereof, which during use of the device is located closest to the medicament delivery site of the patient.

The aim of the present invention is to remedy the drawbacks of the state of the art devices. This aim is obtained by a drive mechanism having the features of the independent patent claim. Preferable embodiments of the invention form the subject matter of the dependent patent claims.

The present invention relates to a drive mechanism for a medicament delivery device that comprises a plunger rod arranged to act on a stopper arranged in a medicament container. The drive mechanism also comprises a compression drive spring operably connected to the plunger rod and capable of applying a force on the plunger rod.

Further a spring guide member is arranged coaxial with, and radially outside, the compression drive spring, capable of preventing buckling of the drive spring. In this manner the spring guide member will very effectively keep the compression drive spring straight even when it is held in a tensioned state, while not making the medicament delivery device unnecessarily long.

The spring guide member has a further feature in that it may comprise a resilient force element capable of applying a force on the medicament container. The force element will then aid in holding the medicament container in place in a medicament delivery device.

In order to function well as a guide for the drive spring, the spring guide may comprise a tubular body extending along a length of said compression drive spring.

The design provides extended guide surfaces inside the tubular body. Preferably the compression spring is arranged coaxially with and radially outside the plunger rod. This provides a somewhat different solution than is normally used where the drive spring is positioned inside a hollow plunger rod and wherein a thin guide rod is placed inside the drive spring.

According to a preferred solution, the resilient force element may comprise a spring guide member spring arranged between a proximally directed surface of said spring guide and a distally directed surface of the medicament container. With this solution, the spring guide member spring may be arranged coaxial with, and radially outside the compression drive spring. Then, in order for the springs not to be engaged with each other, the spring guide member spring may have a winding direction that is opposite the winding direction of the compression drive spring.

The drive mechanism is preferably used in a medicament delivery device comprising a housing, which housing is arranged to accommodate said drive mechanism and said medicament container.

These and other aspects of, and advantages with, the present invention will become apparent from the following detailed description of the invention and from the accompanying drawings.

### BRIEF DESCRIPTION OF DRAWINGS

In the following detailed description of the invention, reference will be made to the accompanying drawings, of which

FIG. 1 is an exploded view of a medicament delivery device comprising a drive mechanism according to the present invention,

FIG. 2a is a detailed view of a housing comprised in the medicament delivery device of FIG. 1,

FIG. 2b is a detailed view of a housing comprised in the medicament delivery device of FIG. 1,

FIG. 3a is a cross-sectional view of the medicament delivery device according to FIG. 1,

FIG. 3b is a cross-sectional view of the medicament delivery device according to FIG. 1,

FIG. 4 is a detailed view of components of the disclosure,

FIG. 5 is a detailed view of components of the disclosure,

FIG. 6 is a detailed view of components of the disclosure,

FIG. 7 is a detailed view of components of the disclosure,

FIG. 8 is a detailed view of components of the disclosure,

FIG. 9 is a detailed view of components of the disclosure,

FIG. 10 is a cross-sectional view of a functional state of the medicament delivery device of FIG. 1,

FIG. 11 is a cross-sectional view of a functional state of the medicament delivery device of FIG. 1,

FIG. 12 is a partial exploded view of a medicament delivery device,

FIG. 13 is a partial cross-sectional view of a medicament delivery device,

FIG. 14 is a partial exploded view of a medicament delivery device,

FIG. 15 is a partial cross-sectional view of a medicament delivery device,

FIG. 16 is a partial exploded view of a medicament delivery device, and

FIG. 17 is a cross-sectional view of a medicament delivery device.

### DETAILED DESCRIPTION OF THE INVENTION

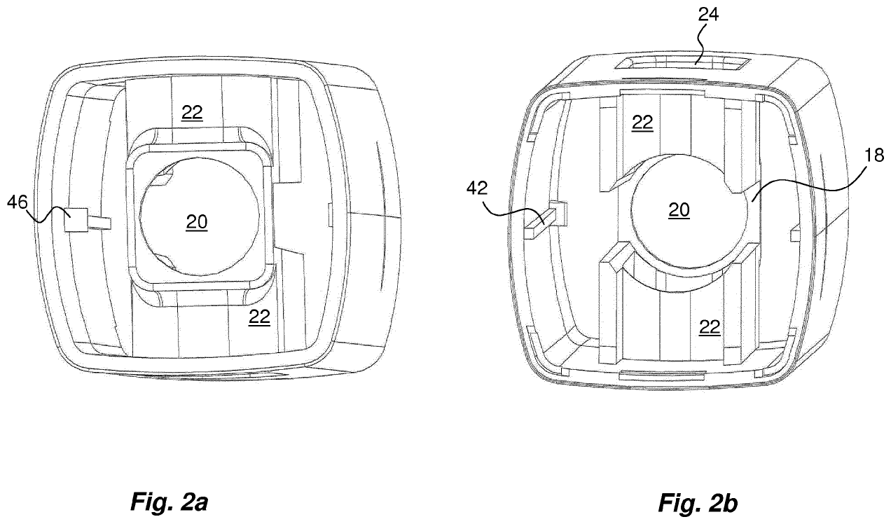

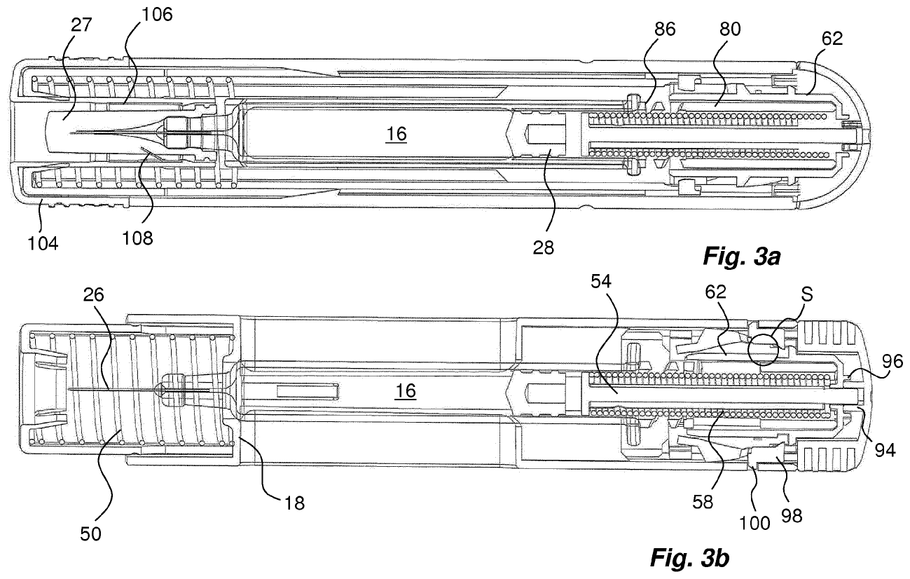

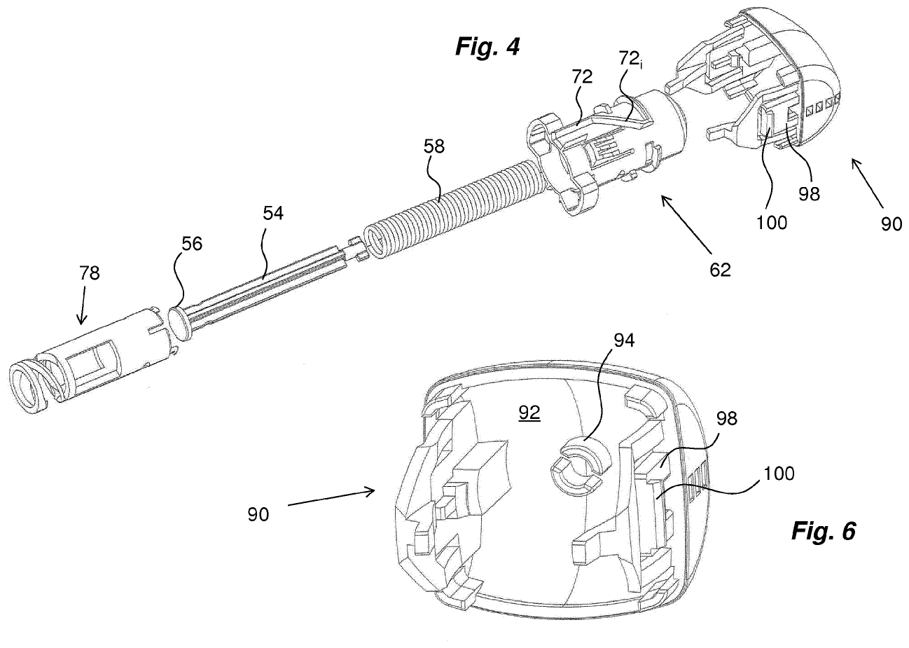

The embodiment shown of a medicament delivery device comprises a generally elongated housing 10 having a proximal end 12 and a distal end 14. The housing 10 is further arranged to accommodate a medicament container 16. Thereby a support surface 18, FIG. 2a and FIG. 2b, is arranged with a central passage 20 through which a proximal end of the medicament container 16 may protrude and wherein a neck portion of the medicament container 16 may rest as seen in FIG. 3b, providing a stop surface in the proximal direction. The support surface 18 is an integral part of wall elements 22 surr-ounding openings or windows 24 in the housing. The wall elements 22 further act as supports for the medicament container 16 in the transversal directions. The medica-ment container 16 is arranged with a suitable medicament delivery member 26 that in the embodiment shown is an integrated injection needle. It is however to be under-stood that other types of medicament delivery members may be used such as attach-able injection needles, wherein different types of attachment elements may be uti-lized such as screw threads, bayonet fittings, luer connections, etc. The medicament delivery member is preferably protected before use by a medicament delivery mem-ber shield 27, in the embodiment shown, a so called rigid needle shield or RNS. It is however to be understood that other types of medicament delivery member shields may be used in order to obtain the desired protection of the medicament delivery member. The medicament container is further arranged with a movable stopper 28.

The proximal end 12 of the housing is arranged with a central passage 30. A medicament delivery member guard 32 is arranged to fit into the central passage 30 and to be movable in the longitudinal direction. The medicament delivery member guard 32 is arranged with a proximal end wall 34 having a central passage 36. The medicament delivery member guard 32 is further arranged with two distally directed arms 38 that run along inner surfaces of the housing. Each arm 38 is arranged with a longitudinal groove 40, in which a guide ledge 42, FIG. 2b, arranged on the inner surface of the housing, may fit. Further, a longitudinal slit 44 is arranged on the arms 38, in which a stop ledge 46, FIG. 2a, may fit. At a distal end of the arms 38, inwardly directed protrusions 48 are arranged, the function of which will be explained. A medicament delivery member guard return spring 50 is further arranged between a proximally directed surface of the wall parts 22 and a distally directed surface of the end wall 34 of the medicament delivery member guard 32.

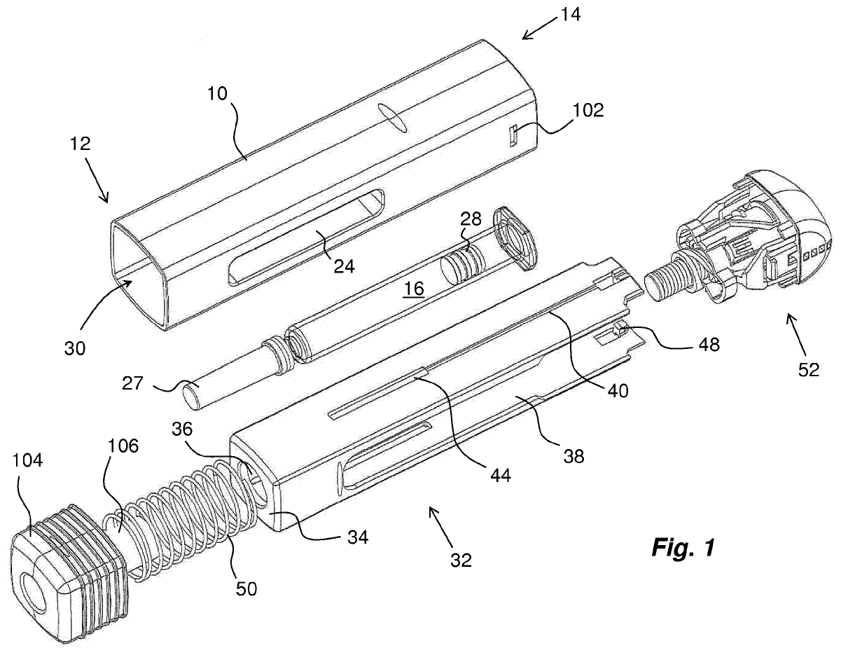

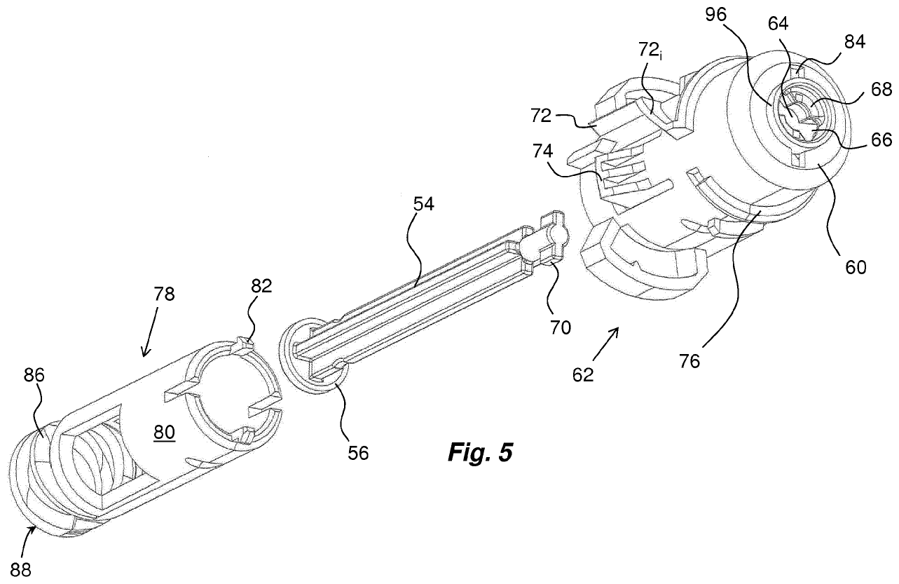

The medicament delivery device is further arranged with a power unit 52, FIGS.1 and 5. It comprises a generally elongated plunger rod 54 provided with a disc 56 at its proximal end, which disc 56 has a diameter somewhat smaller than the inner diameter of the medicament container 16 and which disc 56 is to be in contact with the stopper 28 of the medicament container 16. The plunger rod 54 is surrounded by, and coaxial with, a compression drive spring 58 having its proximal end in contact with a distally directed surface of the disc 56 of the plunger rod 54, and with its distal end in contact with a proximally directed surface of an end wall 60 of a generally tubular rotator 62, FIG. 5.

The end wall 60 of the rotator 62 is arranged with a central passage 64 through which the plunger rod 54 may extend. The passage 64 is further arranged with cut-out sectors 66 with land sectors 68 in between. The plunger rod 54 is in that respect arranged with radially extending wings 70 at its distal end, which wings 70 fit into the cut-out sectors 66 and can rest on the distally directed surfaces of the land sectors 68, as will be described.

The rotator 62 is further arranged with guide ridges 72 that are intended to cooperate with the protrusions 48 of the medicament delivery member guard 32 as will be described, wherein some sections 72, of the guide ridges are inclined in relation to the longitudinal direction of the device. The rotator 62 is further arranged with wedge-shaped protrusions 74 arranged on generally radially flexible tongues, positioned adjacent the guide ridges 72, which wedge-shaped protrusions 74 are intended to lock the medicament delivery member guard 32 after completed use of the device as will be described. The rotator 62 is further arranged with blocking elements 76 that in the embodiment shown is designed as circumferentially extending ledges on the outer surface of the rotator 62, FIG. 5. The ledges 76 have a certain extension and position along the circumference, the function of which will be described below.

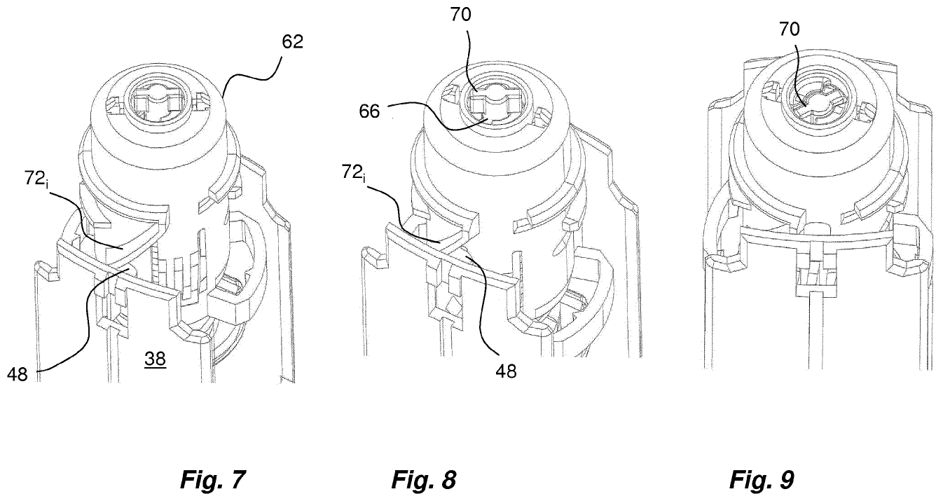

Positioned inside and coaxial with the rotator 62 is a spring guide 78, FIG. 5, having a generally tubular body part 80. A distal end surface of the body 80 is arranged with distally directed hooks 82, which are arranged to fit into passages 84 in the end wall 60 of the rotator 62, for attaching the spring guide 78 with the rotator 62. At the proximal end of the spring guide 78, a compression spring 86 is arranged, either attached to or made integral as seen in FIG. 7. The compression spring 86 is arranged with a ring-shaped end surface 88, which is intended to be in engagement with a distal end surface of the medicament container 16, urging the latter in the proximal direction against the seat 18 of the housing, as seen in FIG. 3a and FIG. 3b.

The medicament delivery device further comprises an end cap 90, FIG. 6, to be attached to the distal end of the housing. The end cap 90 has a dome-shaped end wall 92 where its proximally directed inner surface is arranged with two arc-shaped proximally extending protrusions 94 arranged such that the distal end of the plunger rod 54 with its wings 70 will engage, whereby the plunger rod 54 is rotationally locked. The arc-shaped protrusions 94 also function as guide elements together with a distally directed annular protrusion 96 on the end wall 60 of the rotator 62, providing a journal support as will be described below. The end cap 90 is further arranged with two proximally directed arms 98. Each arm 98 is arranged with a generally radially outwardly extending ledge 100, where the ledges 100 are to engage in recesses 102, FIG. 1, at the distal end of the housing 10 for attachment of the end cap 90 with the housing.

Finally, the medicament delivery device is arranged with a safety cap 104, FIGS. 1 and 3a. It comprises a generally tubular grip body designed to fit onto the proximal end of the medicament delivery device with a friction fit with the medicament delivery member guard 32. A generally tubular medicament delivery member shield remover 106 extends into the central passage of the medicament delivery member guard 32 and surrounds the medicament delivery member shield 27. The medicament delivery member shield remover 106 is arranged with proximally directed inclined tongues 108 that engage with the outer surface of the medicament delivery member shield 27 to grip it.

The device is intended to function as follows. Before delivered to a user, the end cap 90 is not yet attached and a medicament container 16 is entered into the housing 10 from the distal end 14. Thereafter the power unit 52 is placed inside the housing 10. The compression drive spring 58 is in a tensioned state with the plunger rod 54 held in place in relation to the rotator by the wings 70 engaging the land sectors 68. The end cap 90 is attached by pushing it in the proximal direction into the distal passage of the housing 10. The arms 98 of the end cap 90 will flex inwards when the outwardly directed ledges 100 come in contact with the housing 10. They can flex inwards due to the space S between the inner surface of the housing 10 and the outer surface of the rotator 62 as seen in the circle of FIG. 3b. Finally the protective cap 104 is attached to the proximal end of the medicament delivery device. The medicament delivery device may now be delivered to a user.

When a user is to administer a dose of medicament the protective cap 104 is pulled in the proximal direction. Due to the engagement of the medicament delivery member shield remover 106 with the medicament delivery member shield 27, it will be removed from the medicament delivery member 26. The next step is to press the proximal part of the medicament delivery device against a dose delivery site. This will cause the medicament delivery member guard 32 to be moved in the longitudinal direction in relation to the rest of the medicament delivery device, whereby a penetration of the medicament delivery member 26 is performed. The relative movement of the medicament delivery member guard 32 will cause the protrusions 48 to slide along the guide ridges 72 of the rotator 62 such that the protrusions 48 will come in contact with the inclined sections 72, of the guide ridges, FIG. 7, which will cause the rotator 62 to turn around the longitudinal axis of the medicament delivery device, FIG. 8.

The turning of the rotator 62 will cause the cut-out sectors 66 of the passage 64 at the end wall 60 to move in relation to the wings 70 of the plunger rod 54, wherein the plunger rod 54 is prevented from turning due to the engagement with the arc-shaped protrusions 94 of the end cap 90.

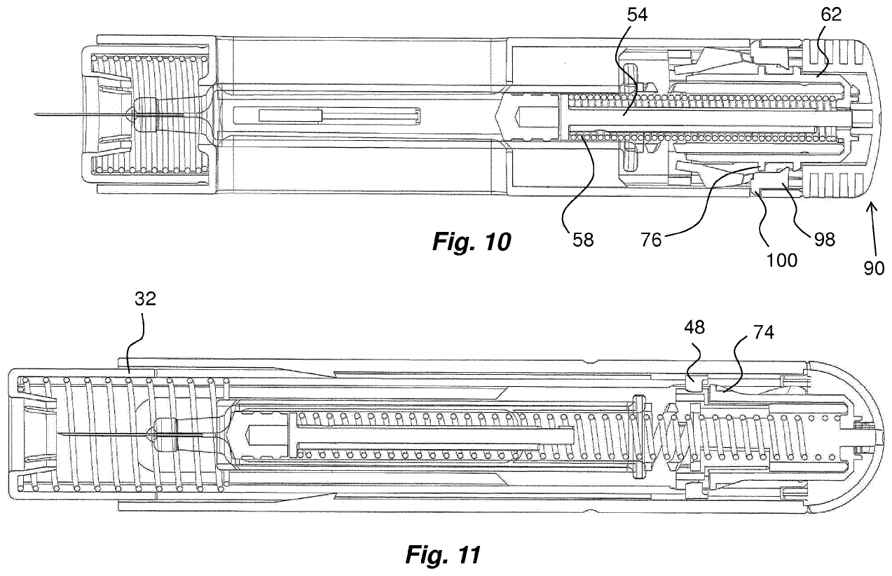

Also, as the rotator 62 is turning, the stop ledges 76 on the outer surface of the rotator 62 are moved adjacent the free ends of the arms 98 of the end cap 90, as seen in FIG. 10. This action will prevent any movement in the radial direction of the arms 98. This aspect is important because when the rotator 62 has turned a certain rotational distance, the cut-out sectors 66 of the end wall 60 of the rotator 62 are aligned with the wings 70 of the plunger rod, FIG. 9, whereby the plunger rod 54 is released, FIG. 10. When the plunger rod is released, so is the compression drive spring 58, which will urge the plunger rod 54 in the proximal direction. However, the drive spring 58 will also exert a force on the end wall 60 of the rotator 62, and thus on the end cap 90 because of the journalled connection of the rotator 62 with the end cap 90, and this force may cause the attachment of the end cap 90 to be disengaged. However, any disengagement is prevented by the stop elements 76 on the rotator 62 effectively preventing any movement of the arms 98.

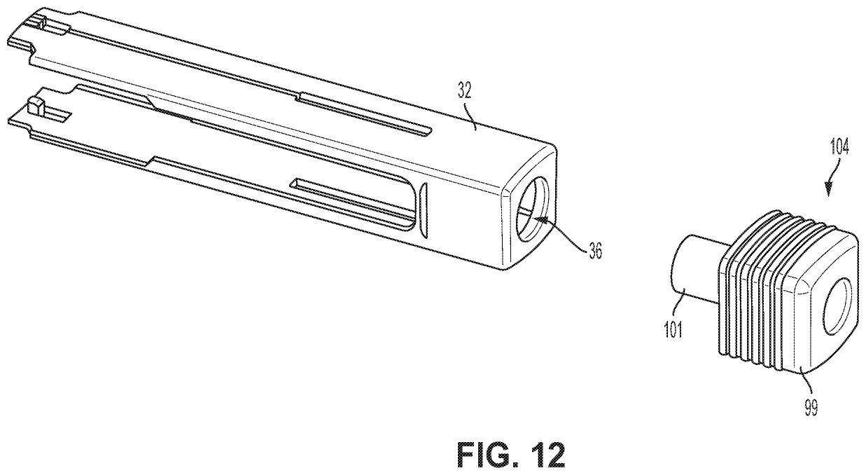

The movement of the plunger rod 54 in the proximal direction will cause it to move the stopper 28, whereby a delivery of a dose of medicament is performed through the medicament delivery member 26. When the dose has been delivered, the medicament delivery device may be removed from the dose delivery site. The medicament delivery member shield 32 will then be moved in the proximal direction by the force of the medicament delivery member shield spring 50, FIG. 11. The movement in the proximal direction of the medicament delivery member shield 32 will cause its protrusions 48 to move over the wedge-shaped protrusions 74 and be positioned proximal of the wedge 74, FIG. 11, thereby locking the medicament delivery member shield 32 in the extended position, thereby covering the medicament delivery member. The device may now be discarded.

FIG. 12 is a partial exploded view of the medicament delivery device, specifically the cap 104 and the member guard 32. The member guard 32 includes the central passage 36. The cap 104 is insertable into the central passage 36 such that a friction fit (e.g., an interference fit) is formed between the member guard 32 and the cap 104. As shown in FIG. 12, the central passage 36 is positioned at a proximal end of the member guard 32. The cap 104 has rotational symmetry with respect to a longitudinal axis of the medicament delivery device. The cap 104 has a body 99 and a tubular extension 101 that extends away from the body 99 along a longitudinal axis of the medicament delivery device. The tubular extension 101 is insertable into the central passage 36 such that the friction fit is formed between the member guard 32 and the tubular extension 101.

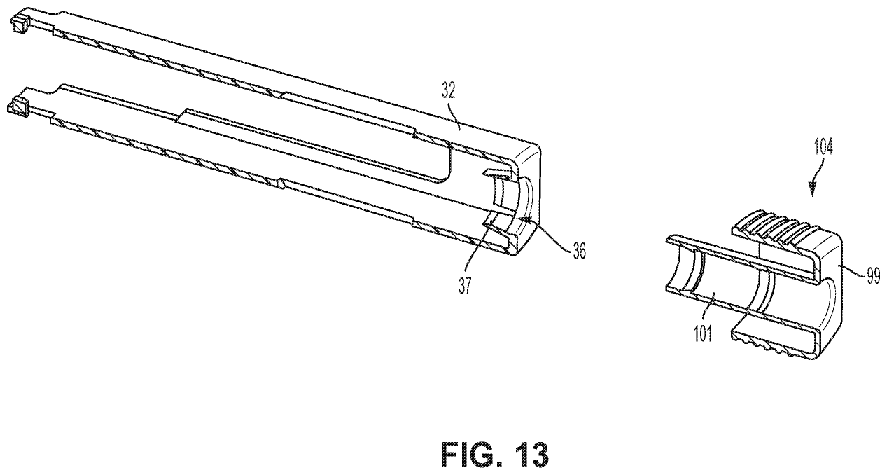

FIG. 13 is a partial cross-sectional view of the medicament delivery device. The member guard 32 includes one or more arms 37 that define the central passage 36. The one or more arms 37 are generally flexible and have rotational symmetry with respect to the longitudinal axis of the medicament delivery device. The cap 104 (e.g., the tubular extension 101) is insertable into the central passage 36 such that the friction fit is formed between the one or more arms 37 and the cap 104 (e.g., the tubular extension 101). The one or more arms 37 extend radially and/or longitudinally inward with respect to the longitudinal axis of the medicament delivery device.

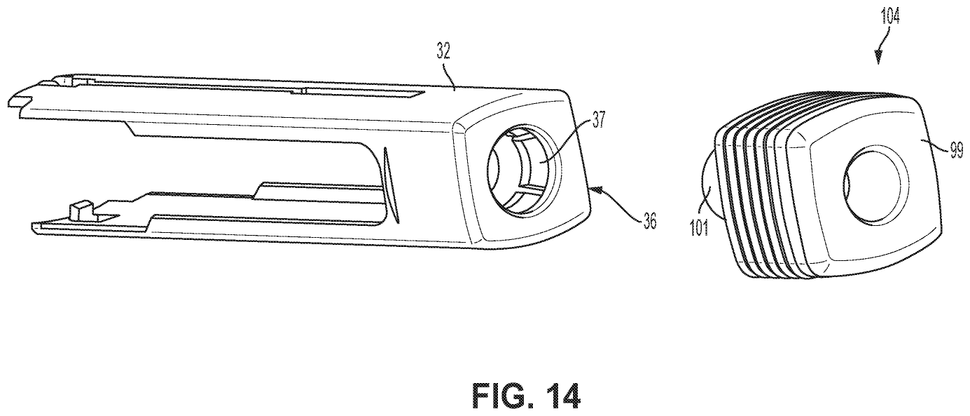

FIG. 14 is a partial exploded view of the medicament delivery device.

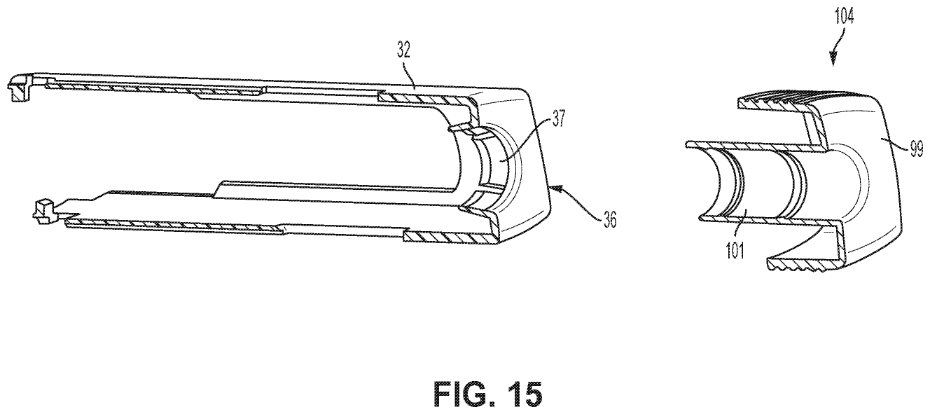

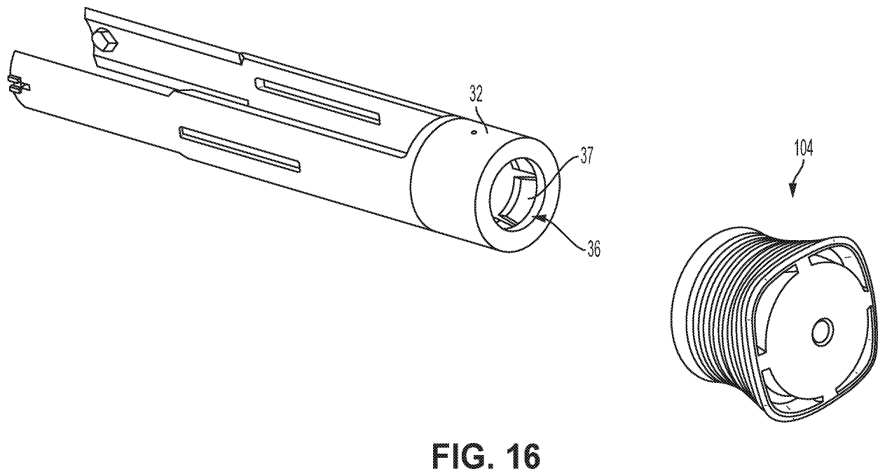

FIG. 15 is a partial cross-sectional view of the medicament delivery device.

FIG. 16 is a partial exploded view of another embodiment of the medicament delivery device.

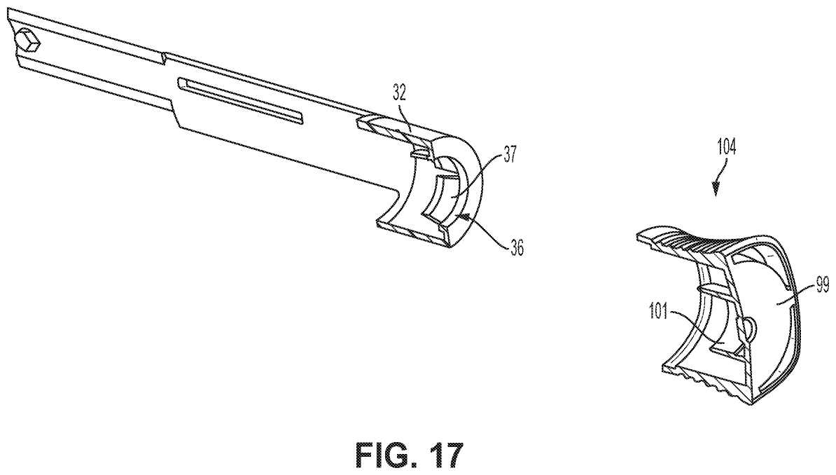

FIG. 17 is a cross-sectional view of the medicament delivery device shown in FIG. 16. In this example, the cap 104 includes a body 99 and a tubular extension 101 that is fully contained within the body 99. The tubular extension 101 is insertable into the central passage 36 such that the friction fit is formed between the member guard 32 and the tubular extension 101.

It is to be understood that the embodiment described above and shown in the drawings is to be regarded only as a non-limiting example of the invention and that it may be modified in many ways within the scope of the patent claims.

1-8. (canceled)

9. A medicament delivery device comprising:
a member guard comprising a central passage, wherein the member guard is configured to house a medicament container; and
a cap that is insertable into the central passage such that a friction fit is formed between the member guard and the cap.

10. The medicament delivery device of claim 9, wherein the member guard comprises one or more arms that define the central passage.

11. The medicament delivery device of claim 10, wherein the cap is insertable into the central passage such that the friction fit is formed between the one or more arms and the cap.

12. The medicament delivery device of claim 10, wherein the one or more arms extend longitudinally inward.

13. The medicament delivery device of claim 10, wherein the one or more arms extend radially inward.

14. The medicament delivery device of claim 10, wherein the one or more arms are flexible.

15. The medicament delivery device of claim 10, wherein the one or more arms have a rotational symmetry.

16. The medicament delivery device of claim 9, wherein the central passage is positioned at a proximal end of the member guard.

17. The medicament delivery device of claim 9, further comprising a shield remover configured to be coupled to the cap, wherein the shield remover is configured for removing a needle shield from the medicament container.

18. The medicament delivery device of claim 9, wherein the cap has a rotational symmetry with respect to a longitudinal axis of the medicament delivery device.

19. The medicament delivery device of claim 9, wherein the cap comprises a body and a tubular extension that extends away from the body along a longitudinal axis of the medicament delivery device.

20. The medicament delivery device of claim 19, wherein the tubular extension is insertable into the central passage such that the friction fit is formed between the member guard and the tubular extension.

21. The medicament delivery device of claim 19, further comprising a shield remover that is configured to be inserted into the tubular extension and configured to remove a needle shield from the medicament delivery device.

22. The medicament delivery device of claim 9, wherein the member guard is configured to move in a distal direction relative to the medicament delivery device during dose delivery via pressing a proximal end of the member guard against a user.

23. The medicament delivery device of claim 22, wherein the member guard is configured to move in a proximal direction relative to the medicament delivery device after dose delivery via moving the proximal end of the member guard away from the user.

24. The medicament delivery device of claim 9, wherein the cap comprises a body and a tubular extension that is fully contained within the body.

25. The medicament delivery device of claim 24, wherein the tubular extension is insertable into the central passage such that the friction fit is formed between the member guard and the tubular extension.

26. The medicament delivery device of claim 24, further comprising a shield remover that is configured to be inserted into the tubular extension and configured to remove a needle shield from the medicament delivery device.

27. The medicament delivery device of claim 24, wherein the member guard is configured to move in a distal direction relative to the medicament delivery device during dose delivery via pressing a proximal end of the member guard against a user.

28. The medicament delivery device of claim 27, wherein the member guard is configured to move in a proximal direction relative to the medicament delivery device after dose delivery via moving the proximal end of the member guard away from the user.
